(19) United States
(12) Patent Application Publication
(10) Pub. No.: US 2004/0082657 A1
(43) Pub. Date: Apr. 29, 2004

(54) Composition for suppressing the appetite of a human being comprising L-theanine and method of administering same

(76) Inventor: Peter Spiegel, Austin, TX (US)

Correspondence Address: SUTHERLAND ASBILL & BRENNAN LLP, 999 PEACHTREE STREET, N.E., ATLANTA, GA, 30309, US

(21) Appl. No.: 10/279,523

(22) Filed: Oct. 24, 2002

(51) Int. Cl. A61K 31/522; A61K 31/195

(52) U.S. Cl. 514/561; 514/263.34

(57) ABSTRACT

The present invention is a method and composition for suppressing the appetite of a human being using L-theanine. The method comprises the step of orally administering a composition comprising an appetite-suppressing amount of L-theanine. The L-theanine composition used as an appetite suppressant in accordance with the invention can be provided in solid form or liquid form and can be further combined with one or more inert ingredients or one or more additional active ingredients. The appetite suppressant composition of the invention provides a natural way of suppressing the appetite of a human being without causing the side effects associated with conventional appetite suppressants.

### FIELD OF THE INVENTION

The present invention relates to a composition for suppressing the appetite of a human being comprising L-theanine and to a method for suppressing the appetite of a human being comprising orally administering a composition comprising L-theanine.

### BACKGROUND OF THE INVENTION

In today's society, people are often less active than their ancestors and spend more time using cars and other vehicles to get around and spend less time walking from place to place. In addition, food and particularly “fast food” is more readily available than it once was. As a result of these societal changes and other factors, the number of people suffering from being overweight or obese has increased in recent times.

In an attempt to keep this problem under control, people are more often turning to diets and appetite suppressants to control their weight. Many of the appetite suppressants that have been on the market in recent years have been found to be unsafe because they produce adverse side effects. Stimulants and thermogenic compounds such as ephedrine, phenylpropanolamine (PPA), amphetamines, fenfluramine and caffeine have been used as appetite suppressants and to increase metabolism and energy levels. However, these compounds typically have undesirable and sometimes dangerous side effects. For example, caffeine, the most common and perhaps the safest compound in this group, can cause dehydration, restlessness, nervousness, gastrointestinal disturbances, muscle twitching, and in some extreme cases, cardiac arrhythmia. Therefore, there is a need in the art for appetite suppressants that do not produce adverse side effects in the body.

### SUMMARY OF THE INVENTION

The present invention provides a method for suppressing the appetite of a human being comprising the step of orally administering a composition comprising an appetite-suppressing amount of L-theanine. It has been discovered that L-theanine works as an appetite suppressant and thus can limit a human being's food intake and weight gain thereby reducing the incidence of overweightedness and obesity. L-theanine also acts as an anti-anxiety agent and thus does not cause the anxiety problems associated with many known appetite suppressants. Furthermore, L-theanine is a natural and healthy compound and does not cause the other side effects associated with many known appetite suppressants.

The L-theanine composition used as an appetite suppressant in accordance with the invention can be provided in solid form or liquid form. The solid form can be a tablet, a pill, a capsule, a powder or a granular substance. The liquid form can be a solution, a suspension or an emulsion. In either solid form or liquid form, the composition typically further includes one or more inert ingredients. Exemplary inert ingredients can be selected from the group consisting of diluents, granulating and disintegrating agents, binding agents, lubricating agents, plasticizers, humectants, solvents, electrolytes, buffers, colorants, aromatic agents, flavoring agents, preservatives, emulsifying agents, compounding agents, formulation agents, permeation enhancers and bulking agents. The method of the invention can also include administering at least one additional compound in combination with L-theanine selected from the group consisting of additional appetite suppressants, amino acids, minerals, vitamins, herbs, nutrients, digestive enzymes, and fat metabolizers. In one embodiment of the invention, the composition can include at least one additional appetite suppressant selected from the group consisting of caffeine, ephedrine, phenylpropanolamine (PPA), amphetamine, methamphetamine, phentermine, fenfluramine, tryptophan, fluoxetine, sertraline, DL-phenylalanine, L-tyrosine, L-glutamine, L-glutamic acid and mazindol. Preferably, the L-theanine is administered in an amount of from about 0.1 mg/kg body weight to about 10.0 mg/kg body weight per day.

The present invention also includes a composition for use in suppressing the appetite of a human being comprising an appetite-suppressing amount of L-theanine. The composition can be in solid form or liquid form and can include inert ingredients and additional active ingredients as discussed above. In one preferred embodiment of the invention, the appetite suppressant compound consists essentially of L-theanine and optionally one or more inert ingredients. In another preferred embodiment of the invention, the composition consists essentially of an appetite-suppressing amount of L-theanine, optionally one or more inert ingredients and optionally one or more additional active ingredients, wherein L-theanine is the only appetite-suppressing compound.

These and other features and advantages of the present invention will become more readily apparent to those skilled in the art upon consideration of the following detailed description, which describes both the preferred and alternative embodiments of the present invention.

### DETAILED DESCRIPTION OF THE PREFERRED EMBODIMENTS

In the following detailed description, preferred embodiments are described in detail to enable practice of the invention. Although the invention is described with reference to these specific preferred embodiments, it will be understood that the invention is not limited to these preferred embodiments. But to the contrary, the invention includes numerous alternatives, modifications and equivalents as will become apparent from consideration of the following detailed description.

The present invention is an appetite suppressant composition comprising L-theanine and a method of administering the appetite suppressant composition. L-theanine is also known as γ-ethylamino-L-glutamic acid and naturally occurs in many types of tea leaves. Theanine (including L-theanine and D-theanine) can be extracted from tea leaves, e.g., with a solvent such as water, hot water or ethanol, or can be prepared by chemical synthesis, microbial fermentation, plant tissue culturing or other processes known in the art. L-theanine is preferably separated from the D-theanine chiral form for use in the compositions of the invention by methods known in the art. Nevertheless, the L-theanine and D-theanine can be included together in the appetite suppressant composition. The L-theanine used in the composition of the invention can be used in a pure form (at least 99% L-theanine), in more crude forms including 50% or more L-theanine, or can be present as a tea extract. The L-theanine can be provided as a pharmaceutically acceptable salt of L-theanine prepared from pharmaceutically acceptable non-toxic inorganic acids and organic acids such as acetic acid, benzenesulfonic acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, p-toluenesulfonic acid and the like.

The L-theanine composition used as an appetite suppressant in accordance with the invention can be provided in solid form or liquid form. In addition to L-theanine, the appetite suppressant composition typically further includes one or more inert ingredients. Exemplary inert ingredients can be selected from the group consisting of diluents, granulating and disintegrating agents, binding agents, lubricating agents, plasticizers, humectants, solvents, electrolytes, buffers, colorants, aromatic agents, flavoring agents, preservatives, emulsifying agents, compounding agents, formulation agents, permeation enhancers and bulking agents.

Exemplary solid forms for oral use include tablets, pills, capsules, powders and granular substances. These solid compositions can include pharmaceutically acceptable inert ingredients such as diluents (e.g. calcium carbonate, sodium chloride, lactose, calcium phosphate, sodium phosphate, and the like); granulating and disintegrating agents (e.g. potato starch, alginic acid and the like); binding agents (e.g. starch, gelatin, acacia and the like); lubricating agents (e.g. magnesium stearate, stearic acid, talc and the like). Other inert ingredients that can be used in the invention include colorants, flavoring agents, plasticizers, humectants and the like. The solid compositions provided in accordance with the present invention can be uncoated or they can be coated by methods and using materials known in the art. In the case of capsules, the L-theanine can be delivered using either a soft gel or a hard gel capsule by mixing the L-theanine with water or an oil medium such as peanut oil, liquid paraffin or olive oil and enclosing the resulting formulation in a capsule.

When a solid form is used, the L-theanine can be delivered over an extended time period by delaying disintegration and absorption in the gastrointestinal tract to provide a sustained release effect. A time delay material such as glycerol monostearate or glycerol distearate can be employed for this purpose. Extended release formulations that can be employed to deliver the active ingredients of the invention are well known in the art.

Exemplary liquid forms for oral use that can be used to deliver the L-theanine of the invention include solutions, suspensions, and emulsions. Preferably, if a liquid form is to be used, an aqueous solution of L-theanine is prepared by dissolving the L-theanine in water. However, the L-theanine can be provided as a suspension by suspending the L-theanine in a solution in admixture with a dispersing or wetting agent, suspending agent and one or more preservatives. Suitable dispersing or wetting agents include naturally occurring phosphatides (e.g. lecithin), condensation products of ethylene oxide, fatty acids, long chain aliphatic acids, partial esters derived from fatty acids and hexitol or hexitol anhydrides (e.g. polyoxyethylene stearate, polyoxyethylene sorbitol monooleate, polyoxyethylene sorbitan monooleate), and the like. Suitable suspending agents are, for example, sodium carboxymethylcellulose, methylcellulose, sodium alginate, and the like. In addition, other inert ingredients can be used in the liquid compositions such as glycols, oils, alcohols, flavoring agents, preservatives, coloring agents and the like.

The method of the invention can also include administering at least one additional compound in combination with L-theanine selected from the group consisting of additional appetite suppressants, amino acids, minerals, vitamins, herbs, nutrients, digestive enzymes, and fat metabolizers. L-theanine is an effective appetite suppressant and is preferably used as the only appetite suppressant in the composition. However, the composition can also include other amino acid appetite suppressants extracted from tea leaves such as L-glutamine, L-glutamic acid, and optionally caffeine. In addition, the composition can include at least one additional appetite suppressant selected from the group consisting of ephedrine, phenylpropanolamine (PPA), amphetamine, methamphetamine, phentermine, fenfluramine, tryptophan, fluoxetine, sertraline, DL-phenylalanine, L-tyrosine and mazindol. Preferably, however, if L-theanine is used with an additional appetite suppressant, the additional appetite suppressant is preferably not a stimulant such as caffeine, ephedrine, amphetamine or methamphetamine.

With respect to the additional active ingredients, exemplary amino acids include those not listed as appetite suppressants above such as alanine, proline, asparagine, L-arginine, L-ornithine, L-aspartic acid, glycine, L-leucine, L-isoleucine, L-valine, L-lysine, L-methionine and taurine. Exemplary minerals include boron, calcium, chromium, copper, fluorine, germanium, iodine, iron, magnesium, manganese, molybdenum, phosphorus, potassium, selenium, silicon, vanadium and zinc. Exemplary vitamins include A, D, E, K, B₁, B₂, B₃, B₆, B₁₂, C, biotin, folic acid and pantothenic acid. The term “herbs” as used herein includes not only herbs but also other botanicals and exemplary herbs and botanicals include garlic, fennel, eucalyptus leaves, Echinacea, St. John's wort, pumpkin seed, hawthorn berry, ginger root, licorice root, chamomile, peppermint, rosemary, dandelion leaf and root, aloe, ginseng, sassafras, sarsaparilla, eleuthero, ashwagandha and ginkgo. Exemplary nutrients include betacarotene, bioflavonoids, choline, inositol and PABA. Exemplary digestive enzymes include protease, amylase and lipase. Exemplary fat metabolizers include chromium picolinate, L-cysteine and L-carnitine.

The L-theanine is administered in a dosage or amount effective to suppress the appetite of a human being. Preferably, the L-theanine is administered in an amount of from about 0.1 mg/kg body weight to about 10.0 mg/kg body weight per day, and more preferably from about 0.5 mg/kg body weight to about 5.0 mg/kg body weight per day. For example, an adult could take 100 mg of L-theanine three times daily or 150 mg of L-theanine twice a day. If the L-theanine is combined with other appetite suppressants, the L-theanine is typically present in a weight ratio of L-theanine to additional appetite suppressant from 1:10 to 10:1 and more preferably from 1:1 to 10:1. In addition to the weight of the human being who is using L-theanine, the preferred dosage can also vary based on the age and response of the individual.

The present invention provides a method for suppressing the appetite of a human being comprising the step of orally administering a composition comprising an appetite-suppressing amount of L-theanine. It has been discovered that L-theanine works as an appetite suppressant and thus can limit a human being's food intake and weight gain thereby reducing the incidence of overweightedness and obesity. L-theanine also acts as an anti-anxiety agent and thus does not cause the anxiety problems associated with many known appetite suppressants. Furthermore, L-theanine is a natural and healthy compound and does not cause the other side effects associated with many known appetite suppressants.

It is understood that upon reading the above description of the present invention, one skilled in the art could make changes and variations therefrom. These changes and variations are included in the spirit and scope of the following appended claims.

That which is claimed:

1. A method for suppressing the appetite of a human being comprising the step of orally administering a composition comprising an appetite-suppressing amount of L-theanine to the human being.

2. The method according to claim 1, wherein the composition is provided in solid form.

3. The method according to claim 2, wherein the solid form is selected from the group consisting of a tablet, a pill, a capsule, a powder and a granular substance.

4. The method according to claim 2, wherein the composition further comprises one or more inert ingredients.

5. The method according to claim 1, wherein the composition is provided in liquid form.

6. The method according to claim 5, wherein the composition further comprises one or more inert ingredients.

7. The method according to claim 5, wherein the liquid form is selected from the group consisting of a solution, a suspension and an emulsion.

8. The method according to claim 1, wherein the administering step comprises orally administering a composition comprising L-theanine and at least one additional active ingredient selected from the group consisting of additional appetite suppressants, amino acids, minerals, vitamins, herbs, nutrients, digestive enzymes, and fat metabolizers.

9. The method according to claim 1, wherein the administering step comprises orally administering a composition comprising L-theanine and at least one additional appetite suppressant.

10. The method according to claim 9, wherein the additional appetite suppressant is selected from the group consisting of caffeine, ephedrine, phenylpropanolamine (PPA), amphetamine, methamphetamine, phentermine, fenfluramine, tryptophan, fluoxetine, sertraline, DL-phenylalanine, L-tyrosine, L-glutamine, L-glutamic acid and mazindol.

11. The method according to claim 1, wherein the composition further comprises at least one inert ingredient selected from the group consisting of diluents, granulating and disintegrating agents, binding agents, lubricating agents, plasticizers, humectants, solvents, electrolytes, buffers, colorants, aromatic agents, flavoring agents, preservatives, emulsifying agents, compounding agents, formulation agents, permeation enhancers and bulking agents.

12. The method according to claim 1, wherein said administering step comprises administering L-theanine in an amount of from about 0.1 mg/kg body weight to about 10.0 mg/kg body weight per day.

13. The method according to claim 1, wherein said administering step comprises administering a composition consisting essentially of L-theanine and optionally one or more inert ingredients.

14. A composition for use in suppressing the appetite of a human being comprising an appetite-suppressing amount of L-theanine.

15. The composition according to claim 14 provided in solid form.

16. The composition according to claim 14 provided in liquid form.

17. The composition according to claim 14, further comprising one or more inert ingredients selected from the group consisting of diluents, granulating and disintegrating agents, binding agents, lubricating agents, plasticizers, humectants, solvents, electrolytes, buffers, colorants, aromatic agents, flavoring agents, preservatives, emulsifying agents, compounding agents, formulation agents, permeation enhancers and bulking agents.

18. The composition according to claim 14, further comprising at least one additional active ingredient selected from the group consisting of additional appetite suppressants, amino acids, minerals, vitamins, herbs, nutrients, digestive enzymes, and fat metabolizers.

19. The composition according to claim 14, consisting essentially of an appetite-suppressing amount of L-theanine and optionally one or more inert ingredients.

20. The composition according to claim 14, consisting essentially of an appetite-suppressing amount of L-theanine, optionally one or more inert ingredients and optionally one or more additional active ingredients, wherein L-theanine is the only appetite-suppressing compound.
